(19) United States
(12) Patent Application Publication
(10) Pub. No.: US 2023/0235415 A1
(43) Pub. Date: Jul. 27, 2023

(54) METHOD OF DETECTION

(71) Applicant: Stemnovate Limited, Cambridge (GB)

(72) Inventors: Ruchi SHARMA, Cambridge (GB); Femi AKANDE, Cambridge (GB)

(21) Appl. No.: 18/007,159

(22) PCT Filed: Jul. 29, 2021
(86) PCT No.: PCT/IB2021/056902

§ 371 Date: Jan. 27, 2023

(30) Foreign Application Priority Data
Jul. 29, 2020 (GB) 2011732.1

(51) Int. Cl. C12Q 1/70; C12Q 1/6855

(52) U.S. Cl. CPC C12Q 1/701; C12Q 1/6855; C12Q 1/6837

(57) ABSTRACT

The present invention relates to a method of detecting specific nucleic acid sequences and a device for performing the method therein. The specific nucleic acid may be prepared from a subject-specimen or from an environmental specimen and the method is performed in isothermal conditions.

### FIELD OF THE INVENTION

The present invention relates to a method of detecting specific nucleic acid sequences and a device for performing the method therein. The invention also relates to a kit of parts.

### BACKGROUND TO THE INVENTION

Methods of detecting specific nucleic acid sequences in a sample often entail the use of expensive equipment and reagents, by highly trained individuals, in a dedicated laboratory setting. This necessitates the transportation of the sample to the laboratory. Given the limitations in equipment, reagent and operator availability, this can lead to significant delays in obtaining the results of the methods.

The most commonly used method is PCR or a variant thereof. PCR uses probes designed to recognise a unique sequence of nucleic acids in a sample by hybridising to that sequence, if present. Rounds of replication of the specific sequence, by repeated heating and cooling of the sample in the presence of unbound nucleic acids, boosts the amount of that specific sequence in the sample. This enables it to be detected by reporter labelling or, more commonly, by gel electrophoresis.

PCR is not only a complex process requiring skilled operatives, it is a relatively slow process and is prone to producing false negative results. In a situation such as the recent COVID-19 pandemic, this can be potentially catastrophic and is a significant limitation in the testing pipeline. Alternative methods, such as antibody testing, are similarly problematic, not least because they are only capable of detecting a response to an infection or a disease, not the infection or disease itself.

Other problems are inherent in the use of PCR in that the reagents are damaging to the environment and, particularly in third world countries, access to testing facilities is simply not available. Furthermore, PCR typically renders the sample unusable for downstream testing, such a sequencing of the positive sample to test for a specific strain of infective agent, for example.

Thus, there is a requirement for a simple to use, accurate, cost-effective and environmentally friendly method for detecting specific nucleic acid sequences. It is amidst this backdrop that the present invention has arisen.

### SUMMARY OF THE INVENTION

In a first aspect there is provided a method of detecting a specific nucleic acid sequence comprising the steps:
a) preparing a population of nucleotide seeker probes and tethering them to a suitable surface, or acquiring a previously prepared and tethered population of nucleotide seeker probes;
b) obtaining or having obtained a specimen, preparing a sample by: extracting nucleic acid material from the specimen and optionally preparing cDNA from the extracted nucleic acid material;
c) contacting the nucleotide seeker probes with the sample under conditions, and for a duration, suitable to permit hybridisation and produce hybridised seeker probes;
d) optionally washing the sample from the hybridised seeker probes to remove unbound nucleic acid material;
e) extending the hybridised seeker probes in the presence of a reporter label, under isothermal conditions and for a duration long enough to produce labelled extended hybridised seeker probes;
f) optionally washing the excess reporter label and nucleotides from the labelled extended hybridised seeker probes; and
g) analysing the sample to identify the presence or absence of labelled extended hybridised seeker probes.

Advantageously, the method enables high fidelity strand-based extension of (nucleotide) seeker probes under isothermal conditions. Advantageously, this removes the requirement for expensive, specialised laboratory equipment such as thermal cyclers.

It is possible for the method to be used in more complex laboratory environments with sophisticated equipment including microfluidics devices such as the one disclosed in WO2020/157697 which is incorporated herein by reference. It is envisioned that, on this device, the seeker probes would be tethered in the culture chamber and the sample and reaction mix flowed through the chamber as required.

In a second aspect there is provided a device suitable for performing the method disclosed herein, the device comprising a plurality of wells, each well having a concave surface suitable for tethering nucleotide seeker probes.

In some embodiments the device is provided with the seeker probes pre-tethered to the suitable surface.

Advantageously, where the seeker probes are pre-tethered, the method can be performed by a less highly technically skilled professional since the method requires obtaining extracted nucleic acid material from a specimen (which could be performed by a different technician and even in a different location) and simply adding the sample containing the nucleic acid material to the wells of the device along with reaction mix and leaving the extension step to occur under isothermal conditions. Since the output of the strand-based extension is a visible reaction, a simple “yes” or “no” answer is easily detected. This also reduces the risk of false positive or false negative results.

Advantageously, the device is scalable and may contain many wells, including suitable numbers for large-scale automation, such as 24 wells, 48 wells, 96 wells, 192 wells, 384 wells, or more or any suitable number.

In a third aspect there is provided a kit of parts for carrying out the method disclosed herein comprising a device according to the present disclosure, a reaction mix, the reaction mix comprising a reporter label and dNTPs suitable for extending the hybridised seeker probes.

The kit can be provided with or without seeker probes which may or may not be pre-tethered according to the requirements of the individual user. Advantageously, the kit may be provided as a “test in a box” whereby the seeker probes are pre-tethered to the device and the user simply adds the sample and reaction mix to the wells of the device.

### BRIEF DESCRIPTION OF THE DRAWINGS

For a better understanding of the invention and to show how the same may be carried into effect, there will now be described by way of example only, specific embodiments, methods and processes according to the present invention with reference to the accompanying drawings in which:

FIG. 1 shows a simplified schematic of the method wherein step 1 entails collecting a specimen and preparing it as a sample by extracting nucleic acid material from the specimen. Step 2 entails preparing cDNA from RNA if necessary or desired. Step 3 entails contacting seeker probes with the nucleic acid material or prepared cDNA (i.e. the sample) to allow hybridisation to occur. Step 4 entails adding reaction mix and incubating under isothermal conditions (e.g. at approximately 72° C. for approximately 60 minutes). At the end of this time a positive or negative result will be detectable.

FIG. 2 shows a diagrammatic representation of the method wherein, in FIG. 2a seeker probes are tethered to a suitable surface and in FIG. 2b hybridisation of sample nucleic acid material to the seeker probes and subsequent extension of the hybridised seeker probes occurs.

FIG. 3 shows a simplified representation of the hybridisation and strand-based extension steps of the method. The seeker probes are contacted with the sample, they hybridise and then the hybridised seeker probes are extended.

FIG. 4 shows a simplified representation of the hybridisation and strand-based extension steps of the method where a bridge seeker probe scaffold is used by anchoring the seeker probe at two points to the suitable surface (e.g. a functionalised solid surface).

FIG. 5 shows a simplified representation of the hybridisation and strand-based extension steps of the method where a prone seeker probe is used by anchoring the seeker probe at multiple points along its length to the suitable surface (e.g. a functionalised solid surface).

FIGS. 6 and 7 show gel electrophoresis results indicating that extension has occurred. In the results shown, RdRP and nucleocapsid seeker probes for nucleic acid sequences specific to SARS-CoV-2

FIG. 8 shows an embodiment of a device for running the method of the present invention featuring ten hexagonal wells. FIG. 8b shows some wells as positive and negative controls and FIG. 8c shows an example of positive and negative results on the device.

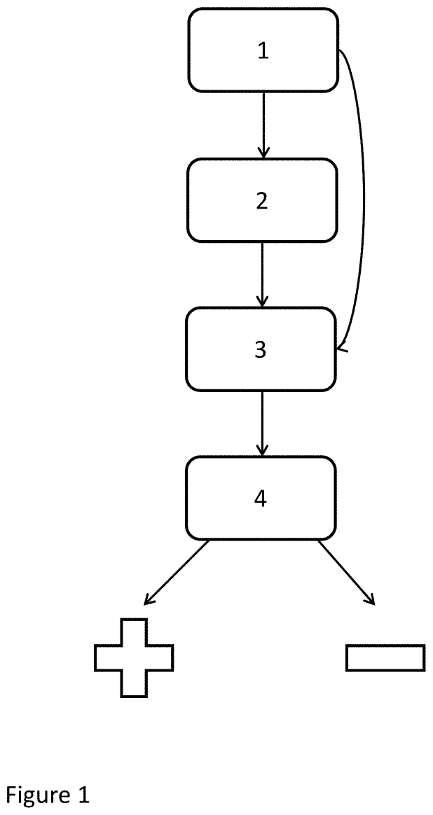

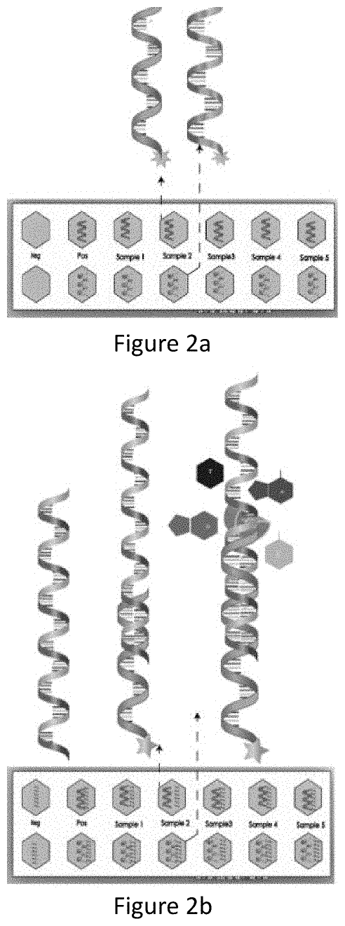

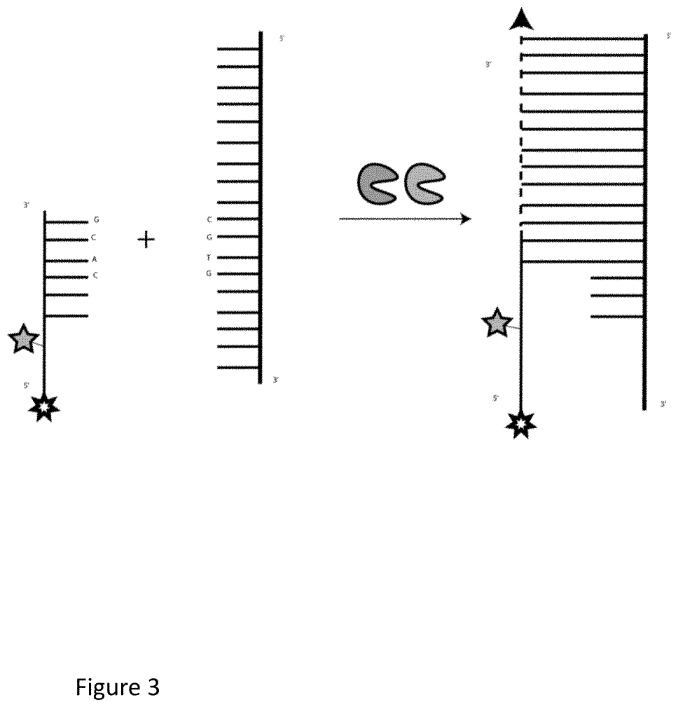

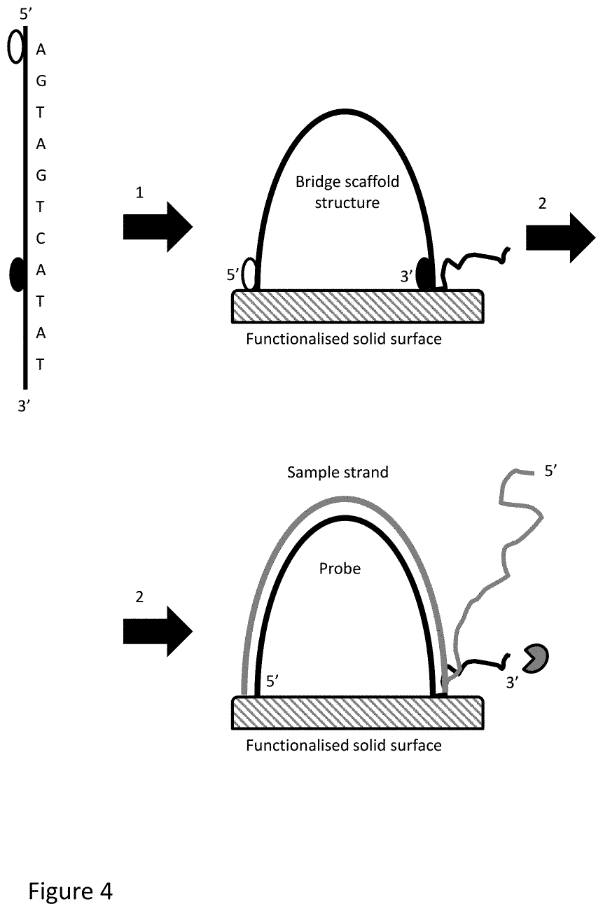

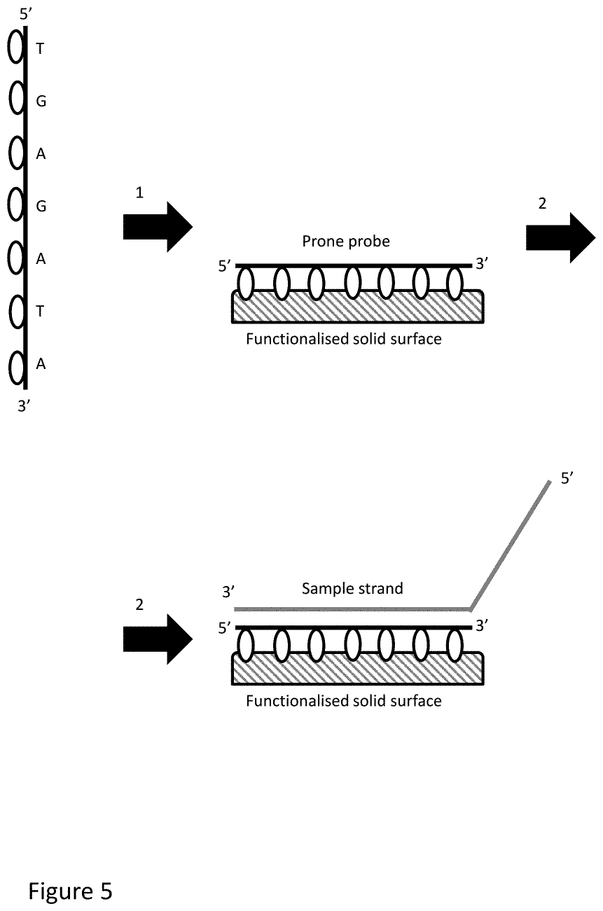

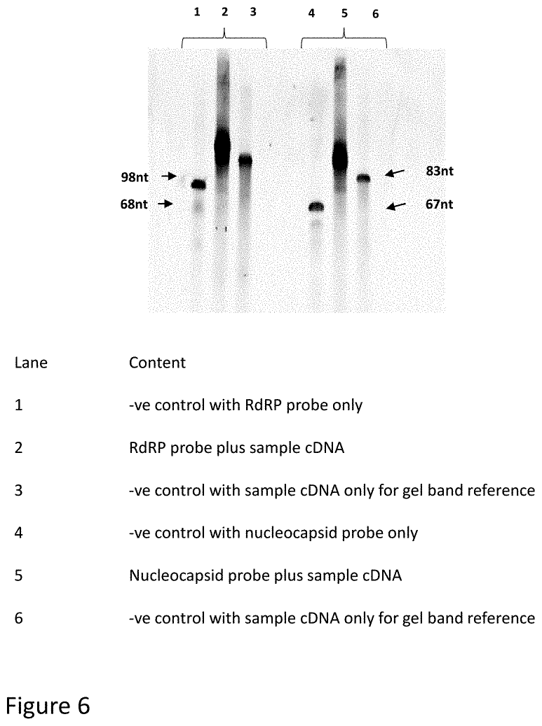

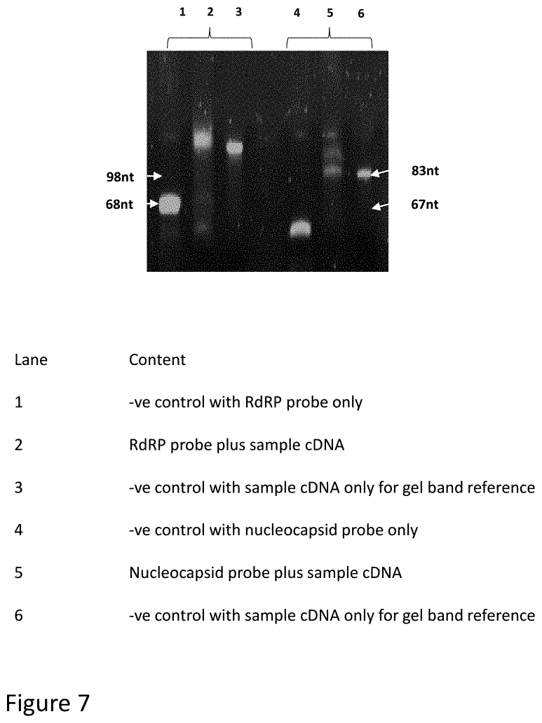

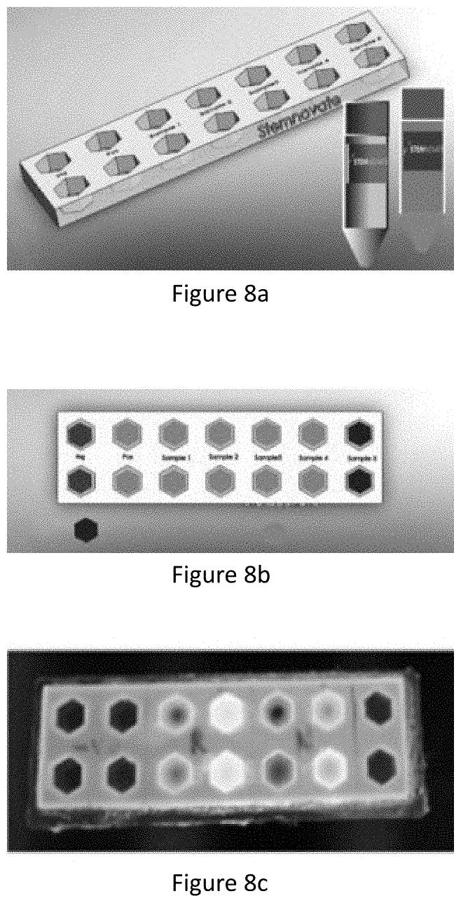

### DETAILED DESCRIPTION

As employed herein specific nucleic acid sequence means any nucleic acid sequence that it is desired to detect in a sample. The nucleic acid may be DNA or RNA and is a sequence capable of correctly and uniquely identifying the sequence to be detected. In some instances the sequence will be conserved across a group of sequences that it is desired to detect. This is also known as the nucleotide or nucleic acid sequence of interest.

As employed herein population of nucleotide seeker probes (seeker probes) means a plurality of at least one nucleotide probe designed to be specific to the nucleotide sequence of interest. In some cases different probes may be designed within the population that are capable of detecting different regions of the nucleotide sequence of interest. In some cases the probes may overlap regions of the sequence of interest. Typically, the seeker probes are single stranded.

In one embodiment the nucleotide seeker probes are DNA seeker probes.

Typically, the seeker probes (SP) are at least 20 nucleotides long.

Typically, the seeker probes are at most 100 nucleotides long.

In one embodiment the seeker probes are 20 to 100 nucleotides long, such at 20, 21, 22, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99 nucleotides long. For example, 55 to 85 nucleotides long. In some embodiments the seeker probes are 65 to 75 nucleotides long.

In some embodiments the seeker probes comprise a cleavage site to permit harvesting of the probes for downstream analysis. Typically, the cleavage site is a specific restriction site. Typically, the cleavage site is located close to the tethered end of the seeker probe. Typically, the cleavage site is located close to the 5′ end of the seeker probe.

In one embodiment the seeker probes are DNA seeker probes. The seeker probes are single stranded to permit hybridisation of the nucleotide sequence of interest.

As employed herein tethering refers to attachment of the seeker probes to a physical substrate/surface.

Tethering may take any suitable form, for example, biotin/streptavidin, thiol/Au bonding, internal amino modifier/Sulfo SANPAH bonding. Typically, the tethering takes the form of covalent bonding.

Typically, the seeker probes are tethered.

In some embodiments the tethering is a single tether at one end, e.g. the 5′ end, of the seeker probe.

In other embodiments more than one tether is used, such as two or more tethers. Where two tethers are used they a typically spaced apart with one tether at or close to the 5′ end and the other tether spaced several nucleotides in the 3′ direction. The two-tether seeker probes form a “bridge probe” scaffold for the sample strand to hybridise to.

In a yet further embodiment, more than two tethers are used, as many as one tether per nucleotide of the seeker probe. These multi-tethered probes are referred to a “prone probes” because they lie prone on the suitable surface. Although the tethering of these probes is more expensive and labour-intensive, these prone probes are reusable by denaturing the hybridised strand and the extended hybridised seeker probe. Advantageously, this means that the detection kit can be used multiple times meaning that the use of material in manufacturing the suitable surfaces and tethering the seeker probes is reduced in the mid to long to term.

Typically, the tethering of seeker probes entails attaching them, in a fixed position, to a suitable surface.

As employed herein suitable surface may be any suitable inert surface, for example, glass or beads, for example, functionalised nanowells, microbeads, surface modified borosilicate glass, metal alloy consisting of or comprising Au, Fe, Pd, Co, Zn and Ti. It will be appreciated that the preparation of the surface to make it suitable to attach the seeker probes will vary depending on the material used.

In one embodiment the seeker probes are tethered on streptavidin dynabeads.

In one embodiment the seeker probes are tethered to the inner surface of a well. That is, the concave surface.

It will be appreciated by the skilled person that strand-based extension according to the method of the invention may be performed without the seeker probes being tethered, provided that the free end of the seeker probe, for example the 5′ end, is blocked with a suitable blocking agent. This method, however, makes downstream collection of the extended hybridised seeker probes difficult.

As employed herein specimen means a small amount of material taken for testing. The specimen may be from a subject, for example a human, animal or plant, such as a blood or tissue specimen; or environmental such a sewage specimen, surface swab or soil specimen. Since nucleic acid can be found in many conceivable specimens, the method disclosed herein is not intended to be limited to a particular source. Therefore, the specimen can be any reasonable, conceivable specimen.

As employed herein sample means the treated and prepared specimen that has been made suitable for using in the disclosed method. For example, the specimen may require dissolving or solubilising, or it may need solid matter removing, cells may need to be lysed, for example. The sample comprises the nucleic acid material extracted from the specimen and presented in a form suitable for using in the method. For example, in nuclease free water. The sample may also be cDNA prepared from the nucleic acid material extracted from the specimen.

As employed herein extracting nucleic acid material means utilising methods known in the art to obtain nucleic acid material from a specimen. For example, lysing cells and extracting nucleic acid from the cells or free in the specimen. For example, those described in “The Protein Protocols Handbook” 3^{rd} edition by John M. Walker (2009) which is incorporated herein by reference.

Preferably the sample contains single stranded extracted nucleic acid material, either RNA or DNA, or cDNA prepared from the RNA.

As employed herein preparing cDNA means complementary DNA. That is, DNA synthesised from single stranded RNA using reverse transcriptase.

As employed herein contacting the seeker probes (SP) with the sample means that the sample is added (or otherwise introduced) to the seeker probes, for example, a solution containing the extracted nucleic acid material (i.e. the sample) is added to the wells in which the seeker probes are tethered or, for example, beads on which the seeker probes are tethered are added to the sample.

As employed herein “under conditions suitable to permit hybridisation” means that the sample and seeker probes are able to anneal into seeker probe/extracted nucleic acid material hybrid, double stranded nucleotide (e.g. DNA). The conditions include the temperature being ambient room temperature, that is approximately 15° C. to 60° C., such as approximately 15, 20, 25, 30, 35, 40, 45, 50 or 55° C., for example approximately 20 to 30° C. (dependent on local climate).

As employed herein “for a duration suitable to permit hybridisation” means the hybridisation step is at least 5 minutes, such as 5 to 10 minutes, or more.

As employed herein hybridised seeker probes means that the seeker probes have selectively bound to the specific nucleic acid sequence of interest where, and only where, that sequence is present in the sample. The seeker probes selectively bind to complementary nucleotide sequences in the sample, where that sequence is present, and do not bind in a non-specific manner. Therefore, if a specific nucleic acid sequence is present in a sample, it will bind to the seeker probes to provide double stranded hybridised seeker probes with overhanging specific nucleic acid sequence, which is single stranded. Where the specific nucleic acid sequence is not present in the sample, no hybridisation will occur.

In order to prevent mismatches and misalignments, conditions can be adjusted, such as salt concentration, according to methods known in the art.

As employed herein washing the sample means rinsing away of excess, unbound nucleic acid material in the sample from the tethered seeker probes, hybridised or otherwise (in the case of a failed or negative reaction) using methods known in the art. For example, washing with 1× Tris-acetate-EDTA (TAE) with 0.2% surfactant.

As employed herein “extending the hybridised seeker probes” (HSP) means that the seeker probe, which is shorter than the specific nucleic acid sequence, is extended using strand-based extension (SBE). That is, nucleic acid of the specific nucleic acid sequence is overhanging and single stranded relative to the bound seeker probe. During the extension step, unbound nucleic acids, (typically in the form of dNTPs) are added to the seeker probe in a stepwise, complementary fashion by an enzyme, typically high fidelity Taq polymerase. The result of this step is that the amount or length of double stranded nucleic acid is increased.

In one embodiment the extension of the seeker probe occurs in a 5′ to 3′ direction.

In one embodiment the extension step begins with the addition of reaction mix to the hybridised seeker probes. Reaction mix may suitably contain all of the reagents required to permit strand-based extension, for example, buffer, enzymes (Taq polymerase), co-factors, dNTPs and reporter label.

As employed herein reporter label means any type of detectable label, such as fluorescent or colorimetric labels or dyes.

As employed herein fluorescent reporter labels include, but are not limited to, TAMRA, Cy3, Cy5, rhodamine red-X, rhodol green, Texas red-X, Oregon green 488/500/514, VIC, Alexa Fluor 488/532/542/555/594/647/750 or FAM.

In one embodiment the reporter label is a colourimetric dye, such as SYBR green I, SYBR green II or SYBR safe. SYBR green non-selectively intercalates with dsDNA, therefore would only be visible where double stranded extension of the hybridised seeker probes has occurred.

As employed herein intercalates means that the reporter label is reversibly bound to the double stranded nucleic acid sequence. Typically, the reporter label attaches to a specific feature of the double stranded nucleic acid sequence, for example the minor groove.

In one embodiment the reporter label is a pH sensitive dye which changes colour as the unbound nucleotides are used up in the extension process, thereby increasing the pH. Where the reporter label is a pH indicator, the reporter label is typically not bound to the extended hybridised seeker probe, thus the “labelled extended hybridised seeker probe” (LEHSP) is not labelled directly, rather the solution around the LEHSP is labelled.

It will be appreciated that there are many ways of utilising reporter labels that are suitable for use with the method disclosed herein. For example, a reporter and quencher system could be employed, wherein the reporter and quencher are present on the seeker probe and one of them is snipped from the seeker probe during the extension step. Once, for example, the quencher is removed from the seeker probe, the reporter is free to fluoresce.

As employed herein isothermal conditions means extension step of the method (step e)) occurs at a constant temperature. Typically the temperature is approximately 70 to 75° C., such as 71, 72, 73 or 74° C. In one embodiment the temperature is 72° C. It will be appreciated that small deviations from the optimum temperature may be tolerated but that a consistent temperature is preferred.

Advantageously, the isothermal extension step means that the method can be carried out with minimal equipment in place and without the requirement for thermal cyclers. For example, a hot plate is sufficient to ensure the method is effective. Similarly, an incubator or water bath could be employed.

As employed herein “for a duration long enough to produce labelled extended hybridised seeker probes” (LEHSP) means that the extension step occurs for long enough to produce a detectable reaction from the reporter label. Typically the duration is less than 120 minutes, such as 30, 40, 50, 60, 70, 80, 90, 100 or 110 minutes. In one embodiment step e) lasts for approximately 60 minutes.

In one embodiment step the extension step, that is step e), lasts for approximately 60 minutes at 72° C.

In one embodiment the hybridisation step and the extension steps are performed concurrently. That is, the reaction mix is added at the same time as the sample nucleic acid material.

In one embodiment the plurality of wells is three or more wells, such as ten wells. It will be appreciated that the device can be scaled accordingly to accommodate as many samples as desired.

In one embodiment the device is made of glass. Advantageously the glass device is environmentally friendly and can be readily recycled or even treated to remove contaminants and re-treated with new probes.

Advantageously the device is amenable to standard laboratory bar coding systems

The sensitivity of the method described herein permits very small volumes of sample and reagents to be used to analyse a sample for a specific nucleic acid sequence. Generally, 20 to 25 microlitre samples are adequate to obtain a reliable result. Furthermore, small amounts of seeker probe are required, in the region of 50 picomoles per well.

As employed herein analysing the sample means comparing the sample to at least one of a positive control and a negative control to determine whether the sample contained the specific nucleic acid sequence of interest.

As employed herein “cleaved from the suitable surface without damaging the labelled extended hybridised seeker probes” or “cleaving the labelled extended hybridised seeker probes from the suitable surface ” means that the seeker probes, hybridised seeker probes or extended hybridised seeker probes (labelled or otherwise) can be easily cleaved from the suitable surface without damage.

In one embodiment the seeker probes are designed with a unique restriction site located at the end closest to the suitable surface (typically the 5′ end). The restriction site permits the seeker probe to be safely detached from the surface by adding the restriction endonuclease to the sample following the extension (and analysis) steps, taking the specific nucleic acid sequence with it. Advantageously, this permits downstream processing of the LEHSPs by sequencing, qPCR, sample library prep, RNA-seq or other techniques.

As employed herein positive control means a control known to give a positive result. In the present method an example of a suitable positive control might be an endogenous house-keeping gene such as beta actin or GADPH. Typically, any suitable gene that is not the nucleic acid sequence of interest but will be present in the sample will be suitable as a positive control.

As employed herein negative control means a control known to give a negative result. In the present method an example of a suitable negative control might contain no seeker probes, therefore no hybridisation can occur. Alternatively, the negative control might contain seeker probes but no dNTPs or polymerase enzyme so that extension cannot occur.

As employed herein lid capable of sealing means a lid that provides a suitable liquid tight seal around each individual well. In one embodiment the lid is an adhesive tape.

Advantageously, sealing the well prevents evaporation during the extension step as well as preventing cross-contamination.

As employed herein disease means any disease or condition that can be detected by identifying the presence of a specific nucleic acid sequence. Envisioned diseases include diseases and conditions caused by genetic mutations, such as cancer, inherited and congenital conditions; diseases caused by infectious agents, such as microorganisms, including viruses, bacteria, fungi, protozoa, helminths and other parasitic organisms.

As employed herein infection means that the disease is caused by an infectious organism.

In one embodiment the infectious organism is a virus.

In one embodiment the virus is a coronavirus.

As employed herein coronavirus means any member of the coronaviridae family, a family of enveloped viruses with a positive-sense single-stranded RNA genome and a nucleocapsid of helical symmetry. In particular, strains of coronavirus that are infectious to humans or non-human animals.

In one embodiment the coronavirus is SARS-CoV-2 or a strain thereof.

As employed herein nucleotide mix means a solution of dNTP nucleic acids, suitable for extending nucleic acid sequence, for example, by Taq polymerase.

The method described herein can be applied on the microfluidic platform as disclosed in patent application WO2020/157697, which is equipped with bespoke automated components that allow controlled flow of chemical reagents, biomolecules or enzymes through single or multiple channels and also possess selective temperature control capability.

Typically, the channel size is approximately 400 μm, channel height is approximately 800 μm and reaction chamber holds approximately 90 μl.

The microfluidic device could be made, for example, of the following components; polydimethylsiloxane, glass, polycarbonate, polyethylene terephthalate or any organic polymer that contains a high content of halogen atoms.

A complete automation cycle includes, for example, priming and flushing the flow cell with buffer. An example method may include the steps: Introducing seeker probes to the culture chamber and permitting them to bind. Alternatively, the chamber may be pre-prepared with seeker probes. Introducing the sample into the culture chamber and permitting hybridisation. Washing the excess sample from the hybridised seeker probes. Introducing nucleotide mix and reporter label to the culture chamber and allowing the extension to occur. Washing the labelled extended seeker probes. Analysing the result. Optionally the bound (tethered) labelled extended seeker probes can be detached from culture chamber and further analysed, for example by sequencing.

For example, it is envisioned that seeker probes could be specifically placed on the culture chamber in an array which may permit use of several types of seeker probe for running in parallel on a single sample. This could be used for identifying causative agents in an unknown infection, for example. Because the device described can be employed with highly sensitive detection methods, both electrodes and microscopic techniques, this level of sensitivity is possible.

Approximately as employed herein means±10%.

In the context of this specification “comprising” is to be interpreted as “including”.

Aspects of the invention comprising certain elements are also intended to extend to alternative embodiments “consisting” or “consisting essentially” of the relevant elements.

Where technically appropriate, embodiments of the invention may be combined.

Embodiments are described herein as comprising certain features/elements. The disclosure also extends to separate embodiments consisting or consisting essentially of said features/elements.

Technical references such as patents and applications are incorporated herein by reference.

Any embodiments specifically and explicitly recited herein may form the basis of a disclaimer either alone or in combination with one or more further embodiments.

### EXAMPLES

### Step by Step Experimental Procedure

### Preparation of Biochip for Probe Attachment

Surface Modification

The chips used was fabricated using the enclosed in-house protocol: glass slides (with dimensions of 76×26 mm was cleaned with absolute ethanol for 15 mins, 2M sodium hydroxide for 30 mins and 0.5 M hydrochloric acid for 30 mins. After each procedure the glass slides were washed in MilliQ water at 130 rpm, 37° C. for 25 mins. Slides were air dried and subsequently treated with UV-Ozone (irradiation at 254 nm). The modified surfaces were confirmed by hydrophilicity test and contact angle upon wetting. Slides were fitted with pre-cleaned and air-dried variable size silicon gaskets (format specific designs). This follows incubation at room temperature with 1% 3-(trimethoxylsilyl) propyl methacrylate (Sigma) and 100 μl of cross-linker methyl acrylate (HA) in ethanol for 6 mins. Silanised slides were then carefully rinsed with absolute ethanol and allowed to dry at room temperature.

Polymer Solution Preparation and Probe Attachment.

The hydrogel on which the functionalised seeker probes was to be covalently linked was synthesized using the following procedure: Prepared 10 to 20% acrylamide solution was degassed for 30 mins. In the same reaction mix, 100 μl of 50 mg/ml to 100 mg/ml ammonium persulphate, and stabilizer was added and quickly vortex and used for polymerization. Polymerisation was carried out on the silanised slides for 1 hr at room temperature. Polymerised spots were washed for 30 mins with MilliQ water after which the silicon gaskets were carefully removed. Silanised glass slides with polymer spots were then left overnight at 37° C. and analysed for spot stability. 50-100 pico moles of functionalised probes were covalently coupled to the prepared spots in PBS for 45 to 60 minutes at room temperature. Non-specifically adsorbed probes were washed off using lx TAE and 0.1% tween-20. Biochips were stored in deionized water at 4° C. for future use.

Fluorescence Dye-Based Amplification Analysis

10 μl of cDNA derived from nasopharyngeal swab specimen, sputum, serum, whole cell lysate and oropharyngeal swabs is applied on the reaction sites (on the biochip) either manually by single or multichannel pipetting or dispensing automatically through a liquid handling system.

Hybridisation of immobilised (tethered) seeker probes to complementary target strands (cDNA) of known predetermined length occurs. Complementary target strands were designed for COVID-19 cDNA's for RNA-dependent RNA polymerase (RDRP), nucleocapsid protein or spike glycoprotein. This step was carried out to minimise background noise (contaminants), false positive occurrence and enrich target material for downstream application.

On-chip strand-based extension (SBE) was carried out at a constant temperature of 72° C. for 60-70 mins with the addition of a 40 μl reaction mix containing high fidelity Taq polymerase enzyme in the presence of deoxyribonucleotide triphosphate (dNTPs), associated cofactors, buffers and an oil interface. Each reaction well on the biochip was covered using an adhesive protective film.

End point reaction readout was monitored by utilising a reporter nucleic acid dye in the presence of blue LED at 450 nm excitation wavelength. A visible colour change was observed with the naked eyes e.g. Red colour indicated negative reaction while green colour indicated positive reaction i.e. an amplification of a SARS-CoV-2 conserved target region.

For subsequent downstream analysis, newly on-chip immobilised SBE polynucleotides can be harvested using cleavage sites present on the probe that permits adaptor attachment and sample library prep. DNA sequencing and bioinformatic analysis can then be carried out on the COVID-19 viral sequences.

The one time use only chips was appropriately discarded with strict adherence to WHO's disposal guidelines.

Formation of Bridge Probe Scaffold

Bridge probes can be achieved by the inclusion of chemical modifications on the backbone of the probe at the terminal 5′ end. The chemical modification on the 3′ end is preferably located at the third to the last nucleotide away from the 3′ end i.e. N-3. This is to permit polymerase strand access and dNTPs incorporation.

These modifications permit a covalent pin down linkage on a functionalised solid surface material. The linkage possesses a high thermodynamic force of greater than 18 kcal mol−1 and has a relatively strong bond. The length of the probe should ideally be greater than >60 mers to encourage hybridization stringency. Strand hybridisation and or extension can be monitored and detected with intercalating dye or fluorescently labelled triphosphates.

Formation of Prone Probe

Prone probes can be achieved with the inclusion of chemical modification on the backbone of the probe. In contrast to the bridge scaffold method, this method of attachment preferably includes the addition of the modification on every (or every other) nucleotide present on the probe. This is a more expensive and labour-intensive approach. An advantage of this method is reusability. After detection, template strand can be conveniently washed off under denaturating conditions, flow cell can be primed and seeker probe can be reused.

1. A method of detecting a specific nucleic acid sequence in a sample comprising the steps:
a) preparing a population of nucleotide seeker probes and tethering them to a suitable surface, or acquiring a previously prepared and tethered population of nucleotide seeker probes;
b) obtaining or having obtained a specimen, preparing a sample by: extracting nucleic acid material from the specimen and optionally preparing cDNA from the extracted nucleic acid material;
c) contacting the nucleotide seeker probes with the sample under conditions, and for a duration, suitable to permit hybridization and produce hybridized seeker probes;
d) optionally washing the sample from the hybridized seeker probes to remove unbound nucleic acid material;
e) extending the hybridized seeker probes in the presence of a reporter label, under isothermal conditions and for a duration long enough to produce labelled extended hybridized seeker probes;
f) optionally washing the excess reporter label and nucleotides from the labelled extended seeker probes; and
g) the analyzing sample to identify the presence or absence of labelled extended hybridized seeker probes.

2. The method according to claim 1, wherein the nucleotide seeker probes are DNA seeker probes.

3. The method according to claim 1, wherein the nucleotide seeker probes are tethered to the suitable surface such that they can be cleaved from the suitable surface without damaging the labelled extended hybridized seeker probes.

4. The method according to claim 3 further comprising the step: h) cleaving the labelled extended hybridized seeker probes from the suitable surface.

5. The method according to claim 1, wherein step c) is carried out room temperature for at least 5 minutes.

6. The method according to claim 1, wherein the reporter label is a fluorescent or colourimetric label.

7. The method according to claim 1, wherein the reporter label is specific to, or intercalates with, double stranded DNA (dsDNA).

8. The method according to claim 7, wherein the reporter label is selected from: SYBR green I, SYBR green II and SYBR safe.

9. The method according to claim 6, wherein the reporter label is a pH indicator.

10. The method according to claim 1, wherein the isothermal conditions of step e) is approximately 72° C.

11. The method according to claim 10, wherein the duration of step e) is approximately 60 minutes.

12. The method according to claim 1, wherein step g) comprises comparing the sample to at least one of a positive control and a negative control.

13. (canceled)

14. The method according to claim 1, wherein there are at least two tethers attaching the nucleotide seeker probe to the suitable surface.

15. The method according to claim 14 wherein the at least two tethers are located at the 5′ end of the nucleotide seeker probe and the 3 nucleotides prior to the 3′ end of the nucleotide seeker probe.

16. The method according to claim 1, wherein there are more than two tethers attaching the nucleotide seeker probe to the suitable surface, such as one tether every other nucleotide or one tether ever nucleotide.

17.-23. (canceled)

24. The method according to claim 1, wherein the specific nucleic acid sequence in the sample is indicative of a disease.

25. The method according to claim 24, wherein the disease is an infection.

26. The method according to claim 25, wherein the infection is a coronavirus.

27. (canceled)

28. The method according to claim 26, wherein the coronavirus infection is a SARS-CoV-2 infection.
